(19) United States
(12) Patent Application Publication
(10) Pub. No.: US 2023/0277103 A1
(43) Pub. Date: Sep. 7, 2023

(54) Integrated Catheter System and Methods for Collection of a Blood Culture Sample

(71) Applicant: Becton, Dickinson and Company, Franklin Lakes, NJ (US)

(72) Inventors: Jonathan Karl Burkholz, Salt Lake City, UT (US); Evan Barbour VandenBrink, San Mateo, CA (US)

(21) Appl. No.: 18/117,043

(22) Filed: Mar. 3, 2023

Related U.S. Application Data
(60) Provisional application No. 63/316,617, filed on Mar. 4, 2022.

(51) Int. Cl. A61B 5/15

(52) U.S. Cl. CPC A61B 5/150992; A61B 5/150213; A61B 5/15074; A61M 2039/1077

(57) ABSTRACT

An integrated catheter is disclosed having a body defining a channel to direct a biological fluid therethrough, a connection port defined in the body, in which an extension tube is fluidly coupled to the connection port, a vented access port in fluid communication with the extension tube, the vented access port spaced apart from the connection port, an integrated catheter operatively connected to the body to direct a biological fluid into the body, and a distal access port defined in the body, the distal access port configured to be in fluid communication with a removable sample collection container. An initial biological fluid volume directed through the body is directed into the extension tube via the connection port and into the vented access port.

### CROSS-REFERENCE TO RELATED APPLICATION

The present application claims priority to U.S. Provisional Application Ser. No. 63/316,617, entitled “Integrated Catheter System and Methods for Collection of a Blood Culture Sample” filed Mar. 4, 2022, the entire disclosure of which is incorporated by reference in its entirety.

### BACKGROUND OF THE INVENTION

### Field of the Invention

The present disclosure is directed to an integrated catheter device and, in particular, a catheter device including a venting access port to redirect an initial volume of biological fluid pulled from a patient.

### Description of Related Art

Catheters are commonly used for a variety of infusion therapies. For example, catheters may be used for infusing fluids, such as normal saline solution, various medicaments, and total parenteral nutrition, into a patient. Catheters may also be used for withdrawing a biological fluid, such as blood, from the patient.

A common type of catheter device includes a catheter that is over-the-needle. As its name implies, the catheter that is over-the-needle may be mounted over an introducer needle having a sharp distal tip. A catheter assembly may include a catheter adapter, the catheter extending distally from the catheter adapter, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Current catheter systems that are used for withdrawing blood from the patient have inherent issues with the quality of the blood culture sample withdraw from the patient. In particular, many current catheter systems collect an initial volume of blood for the blood culture sample, which often may include microbes and other contaminants from the patient's skin that was pierced with the catheter needle. The microbes and contaminants may render the blood culture sample unusable or may provide false test results when analyzing the blood culture sample.

The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

In accordance with an embodiment of the present invention, a catheter with an integrated extension set includes a body defining a channel to direct a biological fluid therethrough, a connection port defined in the body, in which an extension tube is fluidly coupled with the connection port, a distal access port defined in the body, and a vented access port in fluid communication with the extension tube, with the vented access port spaced apart from the connection port. An initial biological fluid volume directed through the body is directed into the vented access port via the extension tube.

In certain configurations, the integrated catheter also includes an integrated extension set operatively connected to the body. The integrated catheter may also include a stabilization platform operatively connected to the body to stabilize the catheter on a desired surface. In other configurations, the integrated catheter extension set may also include a luer adaptor, wherein the vented access port is integrated with the luer adaptor. The device may also include a vent plug integrated with at least one of the luer adapter and the vented access port. In certain configurations, the integrated catheter extension set may include a vent plug operatively connected to the vented access port at an end of the extension tube opposite the connection port.

In certain configurations, the integrated catheter may also include a clamping member disposed about a portion of the extension tube, wherein in a clamping position the clamping member is configured to occlude at least a portion of the extension tube. The integrated catheter may also include a luer lock access device fluidically connected to the distal access port. The vented access port may also include a dual port luer adaptor operatively connected to an end of the extension tube opposite the connection port.

In accordance with another embodiment of the present invention, a catheter with an integrated extension set includes a body defining a channel to direct a biological fluid therethrough, a connection port defined in the body, in which an extension tube is fluidly coupled to the connection port, and a vented access port in fluid communication with the extension tube, with the vented access port spaced apart from the connection port. The integrated catheter also includes an integrated extension set operatively connected to the body to direct a biological fluid into the body, and a distal access port defined in the body, with the distal access port configured to be in fluid communication with a removable sample collection container. An initial biological fluid volume directed through the body is directed into the extension tube via the connection port and into the vented access port.

In certain configurations, a portion of the biological fluid is directed into the removable sample collection container after the initial blood volume is directed into the vented access port. The integrated catheter may also include the removable sample collection container in fluid communication with the distal access port.

In certain configurations, the removable sample collection container is a blood culture sample collection tube. The integrated catheter may also include a stabilization platform operatively connected to the body to stabilize the catheter on a desired surface. Optionally, the integrated catheter extension set can include a luer adaptor, wherein the vented access port is integrated with the luer adaptor. The catheter extension set may also include a vent plug integrated with at least one of the luer adapter and the vented access port. In other configurations, the integrated catheter also includes a clamping member disposed about a portion of the extension tube, wherein in a clamping position the clamping member is configured to occlude at least a portion of the extension tube. The catheter extension set may include a luer lock access device fluidically connected to the distal access port. Optionally, the vented access port may include a dual port luer adaptor operatively connected to an end of the extension tube opposite the connection port.

In accordance with another embodiment of the present invention, a method of collecting a biological fluid sample includes providing an integrated catheter having an integrated extension set having a body defining a channel to direct a biological fluid therethrough, a connection port defined in the body, in which an extension tube is fluidly coupled to the connection port, a vented access port in fluid communication with the extension tube, the vented access port spaced apart from the connection port, and a distal access port defined in the body, the distal access port configured to be in fluid communication with a removable sample collection container. The method also includes receiving an initial volume of the biological fluid in the integrated catheter, and directing the initial volume of the biological fluid through the connection port and into the extension tube fluidly connected to the connection port. The method also includes the step of venting air from the initial volume of the biological fluid through vented access port operatively connected to the extension tube, and occluding the extension tube.

In certain configurations, the method further includes the step of collecting a subsequent volume of biological fluid from the catheter extension set and into a blood culture sample collection tube.

### BRIEF DESCRIPTION OF THE DRAWINGS

Example embodiments will be described and explained with additional specificity and detail through the use of the accompanying drawings in which:

FIG. 1 is a perspective view of a catheter extension set with a blood collection device attached according to one non-limiting embodiment or aspect of the present disclosure;

FIG. 2 is a perspective view of the catheter extension set of FIG. 1 with the blood collection device removed;

FIG. 3 is a perspective view of a catheter extension set with an extension tube for the blood collection device according to one non-limiting embodiment or aspect of the present disclosure; and

FIG. 4 is a perspective view of a catheter extension set with a dual port luer lock adaptor according to one non-limiting embodiment or aspect of the present disclosure.

### DESCRIPTION OF THE DISCLOSURE

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

The use of numerical values in the various ranges specified in this application, unless expressly indicated otherwise, include approximations within the stated ranges. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates, and grammatical variants of those words or phrases.

The figures accompanying this application are representative in nature, and should not be construed as implying any particular scale or directionality, unless otherwise indicated. For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations and step sequences, except where expressly specified to the contrary. Hence, specific dimensions and other physical characteristics related to the embodiments disclosed herein are not to be considered as limiting.

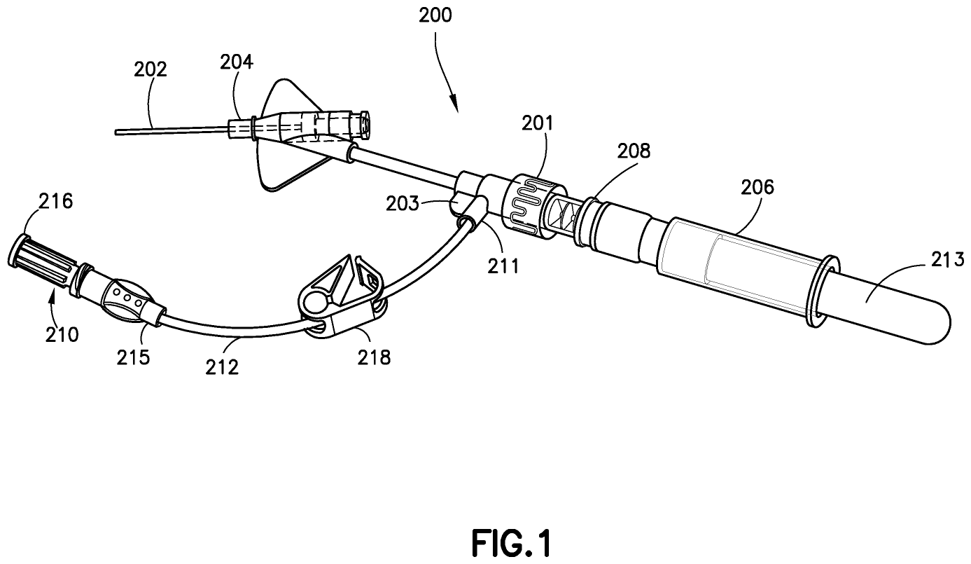

With reference to FIG. 1, according to one non-limiting embodiment or aspect of the present disclosure, an integrated catheter device 200 may be used for extracting a blood culture sample from the patient using a catheter 202 extending from the integrated catheter device 200. In this embodiment, the catheter extension device 200 is integrated with the catheter 202. “Integrated” may be understood to mean that the catheter 202 is operatively connected to or formed integral with the integrated catheter device 200. The catheter 202 may be operatively connected to the integrated catheter device 200 via an adaptor 204 provided on the catheter 202. The catheter extension device 200 may include a body 201 that defines a fluid channel to direct a biological fluid therethrough. In one embodiment, the biological fluid may be blood. A stabilizing platform 203 may be provided on the body 201 to permit the integrated catheter device 200 to be stabilized on the patient's skin.

The integrated catheter device 200 may receive a blood sample through the catheter 202 to direct the blood sample to a luer lock adapter device 206 into which a blood culture sample collection tube 213 may be inserted, as is conventionally known. The integrated catheter device 200 may define a near patient distal access port 208 opposite from the adaptor 204. The distal access port 208 may be configured to mate with the luer lock adapter device 206 that is configured to receive the blood culture sample collection tube 213. The blood culture sample collection tube 213 may be removably inserted into the luer lock adapter device 206 to receive the blood sample that is drawn through the catheter 202. After an adequate blood sample has been drawn from the catheter 202, the blood culture sample collection tube 213 may be removed from the luer lock adapter device 206 in a conventional manner. Multiple blood culture samples may be collected by removing and connecting multiple blood culture sample collection tubes 213 with the luer lock adapter device 206. After all of the desired blood cultures samples and other blood samples have been taken using the integrated catheter device 200, the luer lock adapter device 206 and the blood culture sample collection tube(s) 213 may be disconnected from the distal access port 208 and any remaining blood volume may be flushed from the distal access port 208 or a vented access port 210 (described below) to remove the blood volume from the catheter 202.

In one non-limiting embodiment or aspect, the integrated catheter device 200 may also include a connection port 211 defined between the adaptor 204 and the distal access port 208 of the body 201. An extension tube 212 may be operatively connected to the connection port 211 to receive an initial blood sample from the catheter 202. In one embodiment, as a first volume of blood from the patient enters the catheter 202, the initial volume of blood may be directed through the connection port 211 to fill the extension tube 212 up to the vent feature of the vented access port 210. The vented access port 210 may include a luer adaptor 215 provided on an end of the extension tube 212 opposite the connection port 211. The luer adaptor 215 may be used to operatively and fluidly connect the vented access port 210 to the extension tube 212.

In one embodiment, the vented access port 210 may also include a vent plug 216 held in the vented access port 210. After an initial volume of blood has been isolated through the vent plug 216, a volume of blood may be directed through the distal access port 208 and into the blood culture sample collection tube 213. In one embodiment, an isolated blood collection device may be fluidly connected to the luer adaptor 215 at an end of the extension tube 212, instead of the vent plug 216, to receive and retain the initial volume of blood vented through the vented access port 210. In one aspect, the initial blood volume may be vented to the extension tube 212 to ensure a high-quality blood culture is drawn from the catheter 202 and into the blood culture sample collection tube 213. The initial blood volume received from the catheter 202 may have a higher risk of introducing undesired microbes into the blood culture sample due to bacteria introduced by contact with the patient's skin and dermal layers during the catheter insertion process. In one embodiment of the present disclosure, the initial blood volume that is vented from the integrated catheter device 200 may be greater than 0.15 mL. In one embodiment, the initial volume of blood may occupy only the vent plug 216 or the vent plug 216 and the extension tube 212 between the vent plug 216 and the clamp position of a clamping member 218 (described below).

In one embodiment, when the initial blood volume is directed into the extension tube 212, a blood flashback may be observed in the integrated catheter device 200. During this time, air may be removed from within the extension tube 212 and vented access port 210, which may include vent plug 216.

In one embodiment, the clamping member 218 may be provided on the extension tube 212 to occlude the extension tube 212 and isolate the initial blood volume in the extension tube 212 and/or the vent plug 216 to prevent the initial blood volume from flowing back into the catheter extension device 200. After the clamping member 218 occludes the extension tube 212, the vent plug 216 may be removed from the luer adaptor 215 for disposal of the initial blood volume.

Current methods for extracting a blood culture sample from a patient include using a non-catheter venipuncture device with a manual or passive blood diversion feature to isolate the initial blood volume captured during the venipuncture of the patient. In contrast, the present integrated catheter device 200 adds the vented access port 210, which in combination with following specific methods and procedural steps, allow the collection of a high quality sample for blood culture testing with significantly minimized risk of sample contamination and a costly false positive blood culture test result. Utilizing the present integrated catheter device 200 and procedural method to collect blood culture samples, the need for additional patient pain, hospital cost, and procedural steps required to collect a blood culture sample are eliminated or greatly reduced. By providing a vented access port 210 on the integrated catheter device 200, a physician or nurse can eliminate the need for the additional steps and contamination risks of collecting a discard sample of blood prior to collecting the blood culture sample.

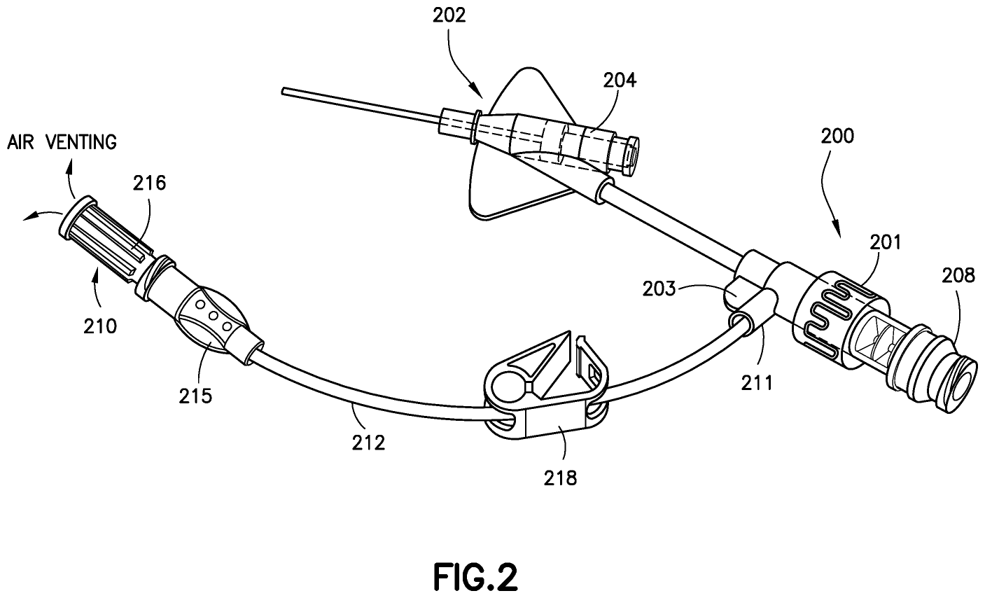

With reference to FIGS. 1 and 2, according to one non-limiting embodiment or aspect of the present disclosure, a method of using an integrated catheter device 200 to draw a blood culture sample from a patient is described. According to the method, a catheter 202 may be first inserted into a patient. The catheter 202 of the integrated catheter device 200 may exhibit an initial flashback of blood including an initial volume of blood is directed through the connection port 211 and into the extension tube 212 upon insertion into the patient. The initial blood volume may be directed to the vent plug 216 held by the luer adaptor 215. The initial volume of blood is directed through the extension tube 212 and may vent air from the extension tube 212 via the vented access port 210, optionally with vent plug 216. A clamping member 218 may then be applied to the extension tube 212 to occlude the extension tube 212 to prevent the initial blood volume from back-flowing into the integrated catheter device 200. After the initial blood volume has been directed through the vented access port 210, the catheter 202 may direct a blood culture sample through the integrated catheter device 200 and out of the distal access port 208 into the blood culture sample collection tube 213. After a desired volume of blood culture samples are received from the distal access port 208, the blood culture sample collection tube 213 may be removed from the integrated catheter device 200 to analyze the blood culture sample.

In one embodiment, during the initial insertion of the catheter 202, an initial blood volume enters the integrated catheter device 200 and the vented access port 210 at the proximal end of the extension tube 212 fluid path due to venous blood pressure. The initial blood volume may enter the integrated catheter device 200 through the catheter 202 which may be connected to the integrated catheter device 200 via an adaptor 204. The initial blood volume may pass through an insta-flash notch defined in the catheter 202 or an interior space between the catheter 202 and a separate flashback structure or needle. The initial blood volume then continues through the catheter 202 and the integrated extension tubing. The blood continues to pass through the integrated extension tubing 212 with the needle in place, as well as after the needle is removed due to the air venting until the blood reaches the vent plug 216.

In one embodiment, when the initial blood volume contacts the vent plug 216, the air venting and the blood flow into the catheter system stops. The vent plug 216 may be a membrane, paper, a porous material, a porous film, or a mechanical feature that allows air to pass, while stopping fluid from passing therethrough. The portion of the blood volume that moves through the vent plug 216, optional adaptor 204, and extension tube 212 is the initial blood volume that enters the integrated catheter device 200 during insertion and is, therefore, at a higher risk of introducing skin flora into the blood culture sample. The integrated catheter device 200 uses a configuration to isolate this initial blood volume to significantly reduce the likelihood of contaminating the blood culture samples with microbes introduced from the skin during the catheter insertion process.

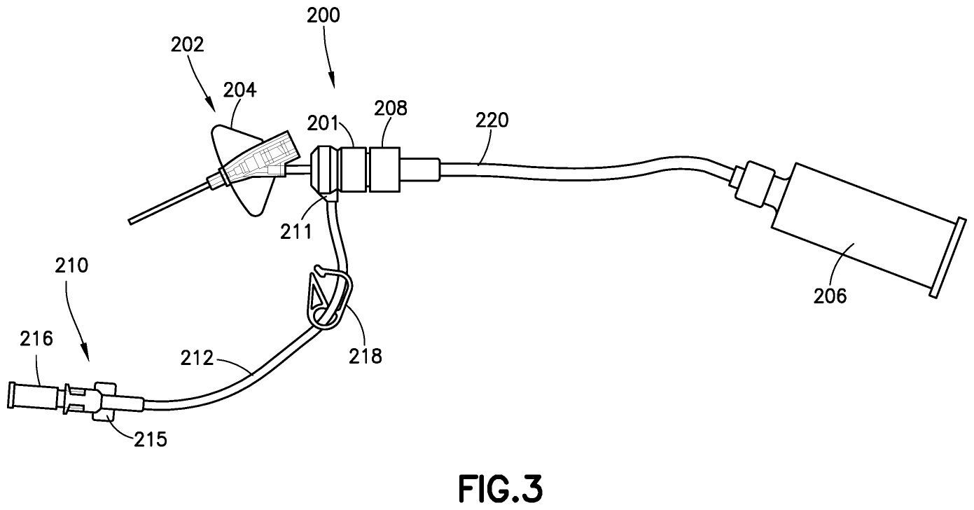

With reference to FIG. 3, in another non-limiting embodiment or aspect of the present disclosure, the luer lock adapter device 206 may be operatively connected to the distal access port 208 via an extension tube 220. The extension tube 220 may be integrated with the luer lock adapter device 206 or removably connected to the luer lock adapter device 206. The extension tube 220 allows for more flexibility during blood collection and the ability to position the blood culture sample collection tube 213 in an upright position during collection. The overall fluid path of the extension tube 220 may be optimized to reduce hemolysis during the blood culture sample collection step and/or subsequent vacuum tube or syringe-based blood samples that are collected after the blood culture sample is collected. In one embodiment, the overall fluid path of the extension tube 220 may include a hemoshield fluid path. In one embodiment, an example of a hemoshield fluid path is disclosed in U.S. Patent Application Publication No. 2021/0186394, published Jun. 24, 2021, the disclosure of which is incorporated by reference in its entirety.

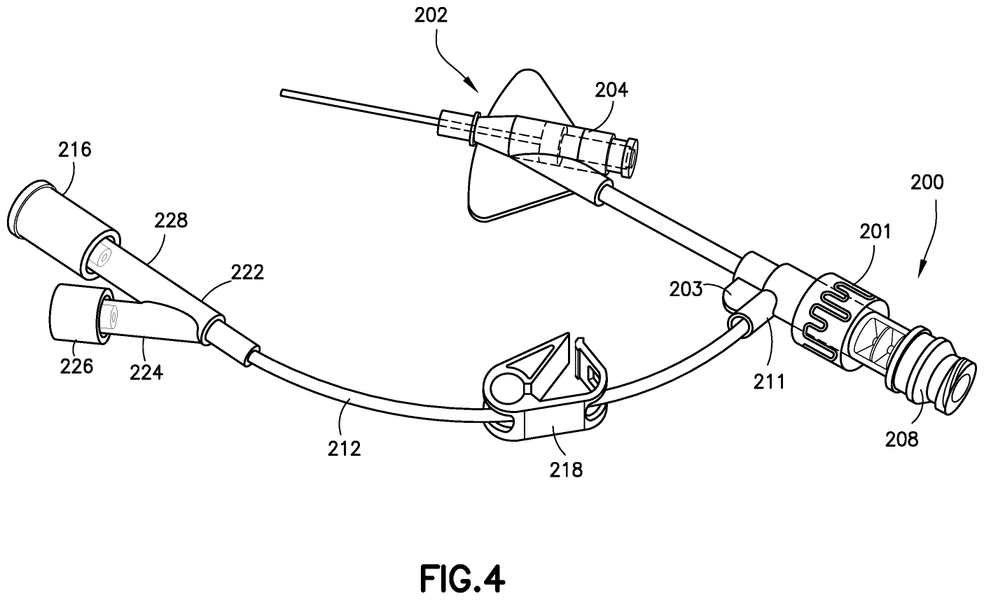

With reference to FIG. 4, in another non-limiting embodiment or aspect of the present disclosure, the extension tube 212 may also include a dual port proximal luer adaptor 222. One port 224 of the dual port proximal luer adaptor 222 may include an end cap 226 for connection to an isolated blood collection device configured to receive the initial volume of blood that is directed through the extension tube 212, while the other port 228 of the dual port luer adaptor 222 may hold the vent plug 216. In some embodiments, the proximal luer adaptor provided on the end of the extension tube 212 may be a single, dual, or triple port. The isolated blood collection device may include a luer lock access device, a luer lock access device with an extension tube, or a luer lock access device with an integrated extension tube to create a sterilized blood collection convenience kit to receive the isolated blood volume from the extension tube 212. In one embodiment, the extension tube 212 may include a pre-attached luer lock access device near the connection port 211 defined in the body 201 of the catheter extension device 200. The dual port proximal luer adaptor 222 may be used with one of the ports vented with the inner volume of the vented port and vent plug 216 are greater than the required isolated blood volume, and the other port 228 being reserved for blood culture sampling. In this type of use, the vent plug 216 wets out and prevents air from entering the system when the blood culture sample is being collected from the other port 228.

In other embodiments or aspects of the present disclosure, the clamping member 218 may be a pinch clamp, a slide clamp, a roller clamp, or a stop-cock style clamp, among other types of clamps. The initial blood volume may be isolated in the extension tube 212 using a one-way venting/fluid valve proximal to the vented access port 210. The initial blood volume may also be isolated in the extension tube 212 using a venting material that wets out and prevents air from enter the integrated catheter device 200 under vacuum during the sample collection step. A feature on the luer lock adapter device 206 or the extension tube 220 may occlude the extension tube 212.

In one embodiment or aspect, in order to ensure an adequate volume of the initial blood sample is diverted and isolated, the internal volume of the vent plug 216 may be greater than or equal to 0.15 mL. Alternatively, the internal volume of the vent plug 216 and the luer adaptor 215 connected to the vented access port 210 may be greater than or equal to 0.15 mL. In another embodiment, an internal volume of the extension tube 212 proximal to a clamp position may be greater than or equal to 0.15 to 2.0 mL. In another embodiment, the internal volume of the extension tube 212 proximal to the clamping member 218 near the vented access port 210 may be greater than or equal to 0.15 to 2.0 mL. In some embodiments, the isolated initial blood volume should be greater than or equal to 0.1 to 5.0 mL.

The present disclosure provides many unique advantages over current blood culture sample collection devices. The number of needlesticks needed for the patient is reduced, resulting in an improved patient experience. The catheter extension device 200 has built in automatic and passive diversion features for capturing an initial blood flow. A reduced risk of contamination for blood culture samples is achieved as all devices used are in sterile packaging. The integrated catheter device 200 may also provide passive blood diversion, including passive or manual (clamping) diverted blood isolation. By diverting the initial blood volume, the need for a blood discard sample is eliminated, as well as the need for a sterilized blood discard sample. Use of the integrate catheter device 200 also reduces the steps in the blood culture sample collection process and improves the workflow of a physician or nurse in which reducing the number of connections made reduces any changes of contamination during the blood culture sample draw. The connection to the luer lock adapter device 206 allows for vacuum tube blood collection immediately after a blood culture sample collection from the patient. The integrated catheter device 200 also provides an optimized fluid path for reduced hemolysis in subsequent blood collection samples.

While the present invention has been described in terms of the above detailed description, those of ordinary skill in the art will understand that alterations may be made within the spirit of the invention.

The invention claimed is:

1. An integrated catheter, comprising:
a body defining a channel to direct a biological fluid therethrough;
a connection port defined in the body, in which an extension tube is fluidly coupled with the connection port;
a distal access port defined in the body;
a vented access port in fluid communication with the extension tube, the vented access port spaced apart from the connection port,
wherein an initial biological fluid volume directed through the body is directed into the vented access port via the extension tube; and
an integrated catheter operatively connected to the body.

2. The integrated catheter of claim 1, further comprising a catheter adapter, wherein the integrated catheter is operatively connected to the body via the catheter adapter.

3. The integrated catheter of claim 2, further comprising a stabilization platform operatively connected to the catheter adapter to stabilize the integrated catheter on a desired surface.

4. The integrated catheter of claim 1, further comprising a luer adaptor, wherein the vented access port is integrated with the luer adaptor.

5. The integrated catheter of claim 4, further comprising a vent plug integrated with at least one of the luer adapter and the vented access port.

6. The integrated catheter of claim 1, further comprising a vent plug operatively connected to the vented access port at an end of the extension tube opposite the connection port.

7. The integrated catheter of claim 1, further comprising a clamping member disposed about a portion of the extension tube, wherein in a clamping position the clamping member is configured to occlude at least a portion of the extension tube.

8. The integrated catheter of claim 1, further comprising a luer lock access device fluidically connected to the distal access port.

9. The integrated catheter of claim 1, wherein the vented access port comprises a dual port luer adaptor operatively connected to an end of the extension tube opposite the connection port.

10. An integrated catheter, comprising:
a body defining a channel to direct a biological fluid therethrough;
a connection port defined in the body, in which an extension tube is fluidly coupled to the connection port;
a vented access port in fluid communication with the extension tube, the vented access port spaced apart from the connection port;
an integrated catheter operatively connected to the body to direct a biological fluid into the body; and
a distal access port defined in the body, the distal access port configured to be in fluid communication with a removable sample collection container;
wherein an initial biological fluid volume directed through the body is directed into the extension tube via the connection port and into the vented access port.

11. The integrated catheter of claim 10, wherein a portion of the biological fluid is directed into the removable sample collection container after the initial blood volume is directed into the vented access port.

12. The integrated catheter of claim 10, further comprising the removable sample collection container in fluid communication with the distal access port.

13. The integrated catheter of claim 12, wherein the removable sample collection container is a blood culture sample collection tube.

14. The integrated catheter of claim 10, further comprising a catheter adapter, wherein the integrated catheter is operatively connected to the body via the catheter adapter.

15. The integrated catheter of claim 14, further comprising a stabilization platform operatively connected to the catheter adapter to stabilize the integrated catheter on a desired surface.

16. The integrated catheter of claim 10, further comprising a luer adaptor, wherein the vented access port is integrated with the luer adaptor.

17. The integrated catheter of claim 16, further comprising a vent plug integrated with at least one of the luer adapter and the vented access port.

18. The integrated catheter of claim 10, further comprising a clamping member disposed about a portion of the extension tube, wherein in a clamping position the clamping member is configured to occlude at least a portion of the extension tube.

19. The integrated catheter of claim 10, further comprising a luer lock access device fluidically connected to the distal access port.

20. The integrated catheter of claim 10, wherein the vented access port comprises a dual port luer adaptor operatively connected to an end of the extension tube opposite the connection port.

21. A method of collecting a biological fluid sample, the method comprising:
providing an integrated catheter comprising a body defining a channel to direct a biological fluid therethrough, a connection port defined in the body, in which an extension tube is fluidly coupled to the connection port, a vented access port in fluid communication with the extension tube, the vented access port spaced apart from the connection port, and a distal access port defined in the body, the distal access port configured to be in fluid communication with a removable sample collection container;
receiving an initial volume of the biological fluid in the integrated catheter;
directing the initial volume of the biological fluid through the connection port and into the extension tube fluidly connected to the connection port;
venting air from the initial volume of the biological fluid through vented access port operatively connected to the extension tube; and
occluding the extension tube.

22. The method of claim 21, further comprising collecting a subsequent volume of biological fluid from the catheter extension set and into a blood culture sample collection tube.
